(19) United States
(12) Patent Application Publication
(10) Pub. No.: US 2024/0315683 A1
(43) Pub. Date: Sep. 26, 2024

(54) REVERSIBLE ELECTROADHESION OF HYDROGELS TO ANIMAL TISSUES FOR SUTURELESS REPAIR OF CUTS OR TEARS

(71) Applicant: University of Maryland, College Park, College Park, MD (US)

(72) Inventors: Srinivasa R. Raghavan, Columbia, MD (US); Leah K. Borden, Silver Spring, MD (US)

(21) Appl. No.: 18/577,826

(22) PCT Filed: Jul. 8, 2022
(86) PCT No.: PCT/US2022/073562

§ 371 Date: Jan. 9, 2024

Related U.S. Application Data
(60) Provisional application No. 63/220,427, filed on Jul. 9, 2021.

(51) Int. Cl. A61B 17/00; C08F 220/56

(52) U.S. Cl. CPC A61B 17/00491; C08F 220/56

(57) ABSTRACT

Electroadhesion, adhesion induced by an electric field, occurs between non-sticky cationic and anionic hydrogels. When gel and tissue are placed under an electric field, the pair strongly adhere, and the adhesion persists indefinitely thereafter. Applying a direct current (DC) field with reversed polarity elimi-nates the adhesion. The use of electroadhesion can seal cuts or tears in tissues or model anionic gels. In an example, electroadhesion works with the aorta, cornea, lung, and cartilage. In another example, electroadhered gel-patches provide a robust seal over openings in bovine aorta, and a gel sleeve is able to rejoin pieces of a severed gel tube.

### CROSS REFERENCE TO RELATED APPLICATIONS

This application claims priority under 35 U.S.C. § 119 to provisional patent application U.S. Ser. No. 63/220,427, filed Jul. 9, 2021. The provisional patent application is herein incorporated by reference in its entirety, including without limitation, the specification, claims, and abstract, as well as any figures, tables, appendices, or drawings thereof.

### TECHNICAL FIELD

The present disclosure relates generally to sutureless tissue repair. In particular, examples of the present disclosure describe, at least, materials and procedures for providing reversible electroadhesion in association with the sutureless repair of tissue.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

Electroadhesion involves two oppositely charged polyelectrolyte hydrogels. Electroadhesion was first reportedly first observed roughly ten years ago. The starting point is to take two solid gels (slabs or strips), each formed by chemical crosslinking of monomers, with one gel having a cationic backbone and the other an anionic backbone. The two gels are contacted with each other along one face and electrodes are placed along either side.

Thereafter a DC voltage is applied in a specific orientation. Within seconds, the two gels become strongly adhered. The same gels will not adhere if contacted in the absence of the field. Thus, the adhesion is induced by the electric field, hence the term ‘electroadhesion’. If the polarity of the field is reversed, the gels lose their adhesion and can be detached.

The mechanism for electroadhesion is still not completely understood, but it is believed to involve molecular rearrangement of both polymer chains and counterions at the gel-gel interface. Thus far, there have been only a few applications of electroadhesion, such as to assemble gels into 3-D structures. On the whole, however, electroadhesion has remained an oddity that has attracted only moderate interest in the scientific community.

Thus, there exists a need in the art for an apparatus which utilize electroadhesion to provide robust seals over openings in cells, tissues (an ensemble of similar cells and their extracellular matrix from the same origin that together carry out a specific function), and organs (a collection of tissues joined in a structural unit to serve a common function).

### SUMMARY

Electroadhesion can be induced between hydrogels and other kinds of soft matter. For example, gels can be electroadhered to animal (bovine) tissues. This result is surprising because while some tissues can be soft and gel-like, they are structurally very different from conventional polymer gels.

Gel-tissue electroadhesion only works between certain types of gels and tissues, and the reasons for the same are disclosed herein. One particularly beneficial application for the gels is to use the gels as an adhesive to reseal damaged tissues. Currently, if a tissue is tom, sutures or staples are needed to rejoin the torn pieces and thereby allow the tear to repair naturally over time. This suturing is a surgical operation that requires considerable skill on the part of a surgeon, and this often implies a difficult and expensive procedure.

Adhesives have been explored as alternatives to sutures during surgery. Several polymeric adhesives are available for surgical use, including those based on cyanoacrylates, fibrin, and polyethylene glycol (PEG) derivatives. Most of these materials are intrinsically sticky and cling upon contact with tissue. Such adhesives have many limitations: in particular, they are usually not strong enough to hold two cut pieces of tissue together. As a result, adhesives usually cannot replace sutures, but are sometimes used along with sutures (e.g., instead of ten sutures, a combination of two sutures and an adhesive may be used). Also, if the adhesive forms a solid film (immediately after application, or after a period of drying), this could result in a physical barrier that hinders the supply of nutrients to the underlying tissue. In comparison, adhesives in hydrogel form are preferable due to their soft nature and their permeability to water and nutrients. For a gel-adhesive to provide a viable alternative to sutures, it should stick strongly to tissues.

Examples on the use of electroadhered gels to seal holes in a tubular animal tissue, i.e., a section of bovine aorta, are demonstrated herein. By applying a DC field of ten volt (10 V) for a short time of between ten and twenty seconds (10-20 s), strong adhesion between gel and tissue is achieved. This adhesion can be reversed at a later time by reversing the polarity of the field. The present disclosure demonstrates the potential utility of electroadhesion in biomedical applications.

The following objects, features, advantages, aspects, and/or embodiments, are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is a primary object, feature, and/or advantage of the present disclosure to improve on or overcome the deficiencies in the art.

It is a further object, feature, and/or advantage of the present disclosure to enable alternative modes of surgery using electroadhesion and obviating the need for sutures. For example, systems of oppositely charged polymer gels, one in the form of rectangular strips and the other as hollow tubes, can be employed. In an initial case, a hole is made in the tube wall and a small gel strip is electroadhered over the hole. Water can flow through the patched tube (with no leaks through the sealed hole) at pressures that exceed normal blood pressure. Next, in a more extreme case, the tube is cut into two and the segments can be rejoined by adhering a sleeve of gel around the cut segments. For this, a long gel strip that is robust and flexible is fabricated, and by sutureless electroadhesion, a similar effect of a suture is achieved, i.e., the cut segments of the tube are joined.

It is still yet a further object, feature, and/or advantage of the present disclosure to expand the landscape of materials that can be electroadhered, and thereby the utility of this electroadhesion.

It is still yet a further object, feature, and/or advantage of the present disclosure to include the ability to achieve adhesion on-command.

It is still yet a further object, feature, and/or advantage of the present disclosure to reverse the electroadhesion in case of error. In other words, the gel patch can be readily detached from the tissue. Subsequently, the patch can be reapplied on command.

It is still yet a further object, feature, and/or advantage of the present disclosure to prophylactically maintain healthy cells and tissue employing the methods described herein, i.e., even in situations where the cells and tissue are not initially damaged.

It is preferred the sutureless methods of repairing tissue be safe, cost effective, and reliable. For example, the hydrogels used herein can be biocompatible (not cause adverse immune response in the body), and the electric fields can be applied in live animals so long as similar voltages (e.g., 10 V DC) are not especially high. Moreover, the field has to be turned on only for a short finite period of time (e.g., 20 s), which is a short enough time to avoid any adverse reaction.

The systems and methods disclosed herein can be used in a wide variety of applications. For example, the potential for electroadhesion to be useful in biomedical scenarios: electroadhesion can enable surgical repairs in the future to be performed without the need for any sutures. Compared to current surgical adhesives, an electroadhered gel patch provides a very robust and durable seal that persists indefinitely. Also, the unique feature of electroadhesion is that electroadhesion develops on command when an external stimulus is applied. Moreover, for many types of surgical repairs, the gel can be biodegraded into benign products within a set number of days after the surgery. Biocompatibility and biodegradability are tractable problems because, in principle, QDM could be replaced with many other kinds of cationic gels.

Methods can be practiced that use, manufacture, and assemble materials that facilitate the repair of tissue and therefore help accomplish some or all of the previously stated objectives.

The materials described herein can be incorporated into systems and kits which accomplish some or all of the previously stated objectives.

These and/or other objects, features, advantages, aspects, and/or embodiments will become apparent to those skilled in the art after reviewing the following brief and detailed descriptions of the drawings. Furthermore, the present disclosure encompasses aspects and/or embodiments not expressly disclosed but which can be understood from a reading of the present disclosure, including at least: (a) combinations of disclosed aspects and/or embodiments and/or (b) reasonable modifications not shown or described.

### BRIEF DESCRIPTION OF THE DRAWINGS

Several embodiments in which the present disclosure can be practiced are illustrated and described in detail, wherein like reference characters represent like components throughout the several views. The drawings are presented for exemplary purposes and may not be to scale unless otherwise indicated.

FIGS. 1A-1B captures gels used in our electroadhesion studies. More particularly, FIG. 1A captures anionic gel of alginate (Alg), crosslinked by divalent Ca²⁺ cations. The gel is made in the form of a hollow tube. FIG. 1B shows a cationic QDM gel strip made by polymerization of acrylamide derivatives. FIGS. 1A-1B show the gel being elastic, stretchable and flexible. Schematics of the gel structure are shown as insets in each case.

FIGS. 2A-2B capture electroadhesion of QDM gel-strip to Alg tube. More particularly, FIG. 2A captures the gel and tube being contacted by graphite electrodes; the positive electrode touching the cationic QDM gel and the negative electrode touching the anionic Alg tube. FIG. 2B shows that upon applying ten volts (10 V) of direct current (DC) for ten seconds (10 s), the gel gets tightly adhered to the tube and conforms to the tube shape. In cases where a puncture is made in the tube wall, adhesion of the gel over the puncture location serves to patch up the puncture, as can be seen in FIGS. 3A-3F.

FIGS. 3A-3F illustrate and capture an electroadhesion of QDM gel used to patch a cut in the Alg tube wall and the changes in pressure during same. More particularly, FIG. 3A is a schematic of the test setup. An aqueous solution of 0.1 wt % FeCl3 is pumped through the lumen of the Alg tube, which is submerged in an aqueous bath of 0.1 wt % tannic acid. If the FeCl3 leaks out of the tube, it reacts with the tannic acid and a black precipitate is formed immediately in the bath. FIG. 3B shows that when the tube is intact, there is no leakage and the bath is clear. In FIG. 3C, the tube is shown punctured with a needle to create a hole of 400 μm diameter. In FIG. 3D, the tube is cut to a length of 7 mm with a blade. In FIGS. 3C-3D, as the fluid in the tube leaks out, the black precipitate can be seen in the bath. FIG. 3E shows the tube from FIG. 3D patched by a QDM gel; when flow is resumed through the tube, no leakage can be seen. FIG. 3F charts results from a pressure gauge placed upstream of the tube records the pressure in the tube. The pressure drops to near-zero in the case of a cut in the tube (similar to FIG. 3D) as the fluid leaks out. When the tube is patched up (similar to FIG. 3E), the pressure is restored to its original value.

FIGS. 4A-4B show pressure changes in tube before and after applying a gel patch by electroadhesion. More particularly, FIG. 4A shows pressure readings before a puncture/cut is made in the wall of an alginate (Alg) tube and after the cut is sealed by electroadhesion of a QDM gel patch. Data are shown for different cut sizes, and for each case, the three bars are the readings for flow (i) before cut (baseline); (ii) when cut is made and not sealed; and (iii) after cut is sealed. In all cases, the pressure drops as fluid leaks out through the cut, but returns to baseline values once the cut is sealed. FIG. 4B shows data related to different cut sizes for the burst pressure required to dislodge the QDM gel patch from the tube wall. For these examples, the baseline pressures are higher than in FIG. 4A. Note that the burst pressure far exceeds the baseline pressure for small cut sizes.

FIGS. 5A-5D illustrate electrical ‘suturing’ of two severed segments of a tube. A long QDM gel-strip is used as a sleeve around the two pieces of the Alg tube. The electrode orientation is as indicated. A schematic of the process is shown in FIG. 5A and the photo in FIG. 5B. Following this process, FIG. 5C shows that the Alg tube segments are found to be ‘sutured’ (joined) by the gel sleeve. Finally, as shown in FIG. 5D, stable flow occurs through the repaired tube to the waste beaker.

FIGS. 6A-6C show electroadhesion of QDM gel to bovine tissue. More particularly, FIG. 6A shows a strip of tissue (T⁻), specifically bovine aorta, and a strip of cationic QDM gel (G⁺) are contacted in a E⁺G⁺T⁻E⁻ configuration, with the gel touching the positive and the tissue the negative electrode. Ten volts (10 V) of direct current (DC) is then applied for twenty seconds (20 s). FIG. 6B shows that this causes the gel to become strongly adhered to the tissue. As shown in FIG. 6C, when the gel-tissue pair is placed in the field with reversed polarity (ET-GE), then within ten seconds (10 s), the adhesion is lost and the gel can be detached from the tissue.

FIG. 7 shows all pairs were placed in an electric field of ten volts (10 V) direct current (DC) that was applied for twenty seconds (20 s). In the gel-gel cases, a cationic QDM gel (G⁺) was contacted with an anionic SA gel (G⁻). In the gel-tissue cases, the cationic QDM gel (G⁺) was contacted with various tissues. In all examples, the current I starts high and decreases with time. The highest recorded current is used to calculate the current densities j shown in the plot (note: j=I/area of contact). The area of contact ranged between 1.6 and 2.4 cm2 for the various tissues. No obvious correlation is seen between j and the occurrence of electroadhesion (refer to Table 1 in the text). The data for gel-gel pairs in their native state (i.e., as prepared in deionized water) or after soaking in PBS demonstrate that j mainly depends on the ionic strength of the fluid.

FIGS. 8A-8C measure adhesion strength using the lap-shear protocol. More particularly, FIG. 8A is a schematic of the lap-shear example and a photo of an example in progress. Samples are first adhered over a lap region and then affixed to glass slides on their reverse sides using cyanoacrylate glue. Tension is then applied to the ends of the slides. FIG. 8B plots stress vs. strain curves from lap-shear examples for two sets of samples: gel-gel (QDM-Alg) and gel-tissue (QDM-aorta). Data are shown for the cases of electroadhesion and contact adhesion (control). The samples delaminate at the end of each curve, marked by an X. The stress at this point is a measure of the adhesion strength. FIG. 8C graphs adhesion strengths from the curves in FIG. 8B for the QDM-Alg and QDM-Aorta samples and for the two cases of electro- and contact adhesion. In each category, at least three samples were analyzed, and the averages are plotted. Error bars correspond to standard deviations.

FIG. 9 shows adhesion between QDM gels and aorta strips were measured after different durations of exposure to the field (10 V DC). The lap-shear technique was used and the stress-at-break was used to quantify the adhesion strength. The plot shows that electroadhesion develops within approximately ten seconds (˜10 s) in the field and the adhesion strength saturates by about twenty seconds (20 s). The data shown are averages (across at least three samples) and the error bars correspond to standard deviations. The line through the data is a guide to the eye.

FIGS. 10A-10D show electroadhesion of QDM gels to patch openings in the aorta. More particularly, FIG. 10A shows the anatomy of the aorta, which is a large artery, is depicted on the left. A 15-cm long segment from the descending thoracic region of the aorta is used in the study. The segment is a hollow tube that has holes on its surface corresponding to arterioles (side branches), as shown both in the schematic and the photo. FIG. 10B shows that when an aqueous solution of 0.1 wt % FeCl3 is pumped through the aorta, the fluid leaks out of the arterioles and falls into the bath containing tannic acid, whereupon a black precipitate of ferric tannate is formed. FIG. 10C shows two QDM gel strips electroadhered to the aorta so as to cover the arterioles. FIG. 10D shows when the FeCl3 solution is pumped through the patched aorta, no leaks are observed (the bath stays clear), and the fluid flows steadily into the beaker on the right.

An artisan of ordinary skill in the art need not view, within isolated figure(s), the near infinite number of distinct permutations of features described in the following detailed description to facilitate an understanding of the present disclosure.

### DETAILED DESCRIPTION

The present disclosure is not to be limited to that described herein. Mechanical, electrical, chemical, procedural, and/or other changes can be made without departing from the spirit and scope of the present disclosure. No features shown or described are essential to permit basic operation of the present disclosure unless otherwise indicated. For example, it is to be appreciated that where the present disclosure discusses adhesion amongst tissues and gels, that similar electroadhesion can apply to cells (a biological organization level lower than tissues) and organs (a biological organization level higher than tissues). In yet another example, it is to be appreciated that where the present disclosure calls for a direct current (DC) to be used, an alternating current (AC) could be used in lieu thereof, unless explicitly stated otherwise.

Gel-gel electroadhesion can be established with a combination of gels, one cationic and the other anionic. To mimic tubular tissues, an anionic gel can be provided in the form of a tube 100 with diameter 102. By way of example, the gel can comprise anionic polysaccharide sodium alginate 104 crosslinked into a network by divalent Ca²⁺ cations 106.

An example procedure for creating tubes 100 with a wall of alginate (Alg) gel is described in by one of the inventors of the present application in Gargava et al., “Rapid electroformation of biopolymer gels in prescribed shapes and patterns: A simpler alternative to 3-D printing. ACS Appl. Mater. Interfaces 11, 37103-37111 (2019)”, which is herein incorporated by reference in its entirety. More particularly, the use of electric fields to rapidly form gels of the biopolymer alginate (Alg) 104 in specific three dimensional (3-D) shapes and patterns is described. For example, a gel of the biopolymer agarose, which is thermoresponsive and hence can be molded into a specific shape. The agarose mold can then be loaded with Ca² cations 106 and placed in a beaker containing an Alg solution. The inner surface of the beaker is surrounded by aluminum foil (cathode 300+), and a copper wire (anode 300−) is stuck in the agarose mold. These are connected to a direct current (DC) power source, and when a potential of approximately ten volts (˜10 V) is applied, an Alg gel is formed in a shape that replicates the mold. Gelation occurs because the Ca²⁺ ions 106 electrophoretically migrate away from the mold, whereupon they cross-link the Alg chains 104 adjacent to the mold. At low Ca²⁺ (0.01 wt %), the Alg gel layer grows outward from the mold surface at a steady rate of about 0.8 mm/min, and the gel stops growing when the field is switched off After a gel of desired thickness is formed, the agarose mold can be melted away to leave behind an Alg gel in a precise shape. Alg gels 100 formed in this manner are transparent and robust. This process is particularly convenient to form Alg gels 100 in the form of hollow tubes, including tubes with multiple concentric layers, each with a different payload. The technique is safe for encapsulation of biological species within a given Alg layer. Alg gels 100 can also be created in specific patterns by directing gel growth around selected regions. This technique enables lab-scale manufacturing of alginate gels in 3-D without the need for an expensive 3-D printer.

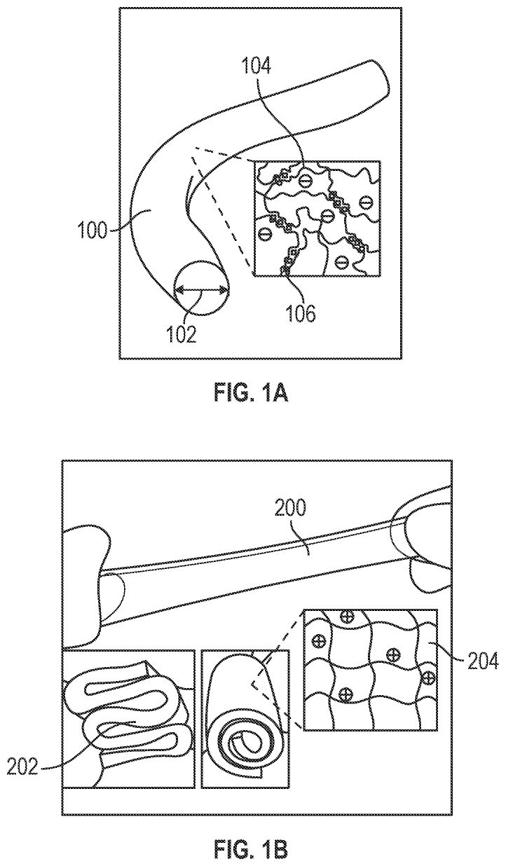

Through this procedure, all the dimensions of the tube 100, including the length, inner diameter 102, and wall thickness. FIG. 1A shows an approximately ten centimeter (˜ 10 cm) long tube with an inner diameter of approximately one centimeter (˜ 1 cm) and a wall thickness of approximately one millimeter (˜ 1 mm). The inset illustrates the structure of the gel wall, which consists of Alg chains 104 connected at zones by Ca²⁺ ions 106. The tube has a pink color due to a trace amount of rhodamine B (RB) dye added during the synthesis.

The counterpart to this tube 100 is a gel 200, made in the form of a rectangular strip. The gel 200 shown has a thickness 202 of approximately two millimeters (˜2 mm) This gel is synthesized by polymerizing a mixture 204 containing acrylamide (AAm, a nonionic monomer), quaternized dimethyl aminoethyl methacrylate (QDM, a cationic monomer), bis(acrylamide) (BIS, a nonionic crosslinker) and laponite (LAP) nanoparticles. The molar ratio of QDM relative to all the monomers dictates the level of charge on the gel strands, and this can be kept at 16 mol %. The ratio of BIS to all the monomers dictates the stiffness of the gel, and this can be maintained at 1.6 mol %. If the BIS content is too high, the gel becomes brittle. Adding 0.1 wt % of LAP to the gelling mixture significantly improves the flexibility and stretchability of the final gel. The overall gel 200 is denoted as QDM 200− to signify its cationic nature. FIG. 1B shows that the QDM gel strip 200− is flexible enough to be twisted or rolled up. The strip 200 can also be stretched up to ˜ 1.75 times its original length without rupture.

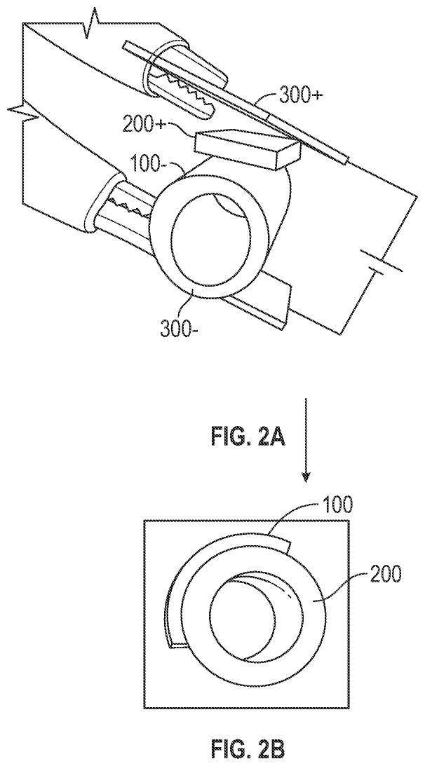

The cationic QDM gel-strips 200− can be electroadhered to the anionic Alg gel-tubes 100. Electroadhesion can involve two graphite electrodes 300 and a DC power supply. The electrodes 300 have to be placed in contact with the above gels in a particular orientation, as shown by FIG. 2A. That is, the strip 200 and tube 100 are brought into contact.

Electrodes 300+, 300− of many different types can work, including but not limited to the graphite electrodes described above, silver electrodes, platinum electrodes, needle electrodes, ring electrodes, and/or any other suitable types of electrical conductors used to make contact with a nonmetallic part of a circuit (e.g., a semiconductor, an electrolyte, a vacuum or air). Electrodes 300+, 300− can be applied from one side of the tissue, e.g., both from the exterior of the cylindrical vessel.

More particularly, the positive electrode (E+) 300+ is made to contact the cationic gel strip (G⁺) 200+, while the negative electrode (E−) 300− is contacted with the anionic gel tube (G⁻) 100. In other words, the cathode 300− (of various shapes) is in contact with the gel 200−, the gel 200− contacts the tissue 100+, and the tissue 100+ contacts the anode 300+(of various shapes). The anode 300+ should not touch the gel 200−.

With this orientation (denoted from now on by E⁺G⁺G⁻E⁻) a DC voltage of ten volts (10 V) can be applied for approximately ten seconds (˜10 s). When the voltage is switched off, the QDM strip 200− is found to be strongly adhered to the alginate tube 100+(FIG. 2B), and this adhesion persists thereafter. If the reverse electrode orientation (E+G−G+E−) is used at the start, the two gels will not stick. Conversely, if electroadhered gels 200− are reconnected to the field in the above reverse orientation, and a ten volt (10 V) field is applied for approximately ten seconds (˜ 10 s), the gels 200− lose their adhesion and can be easily detached. Electroadhesion between a covalently crosslinked gel and a physically crosslinked one is therefore demonstrated. In previous reports of electroadhesion, all gels were covalently crosslinked.

The hydrogels used within the gel strips 200 are crosslinked hydrophilic polymers that do not dissolve in water. The hydrogels are thus able to form a three-dimensional network of hydrophilic polymers holding water. The hydrogels are highly absorbent and therefore maintain well defined structures. These properties underpin several applications, especially in the biomedical technological field. The types of hydrogels employed can be synthetic or in can be derived from nature.

The crosslinks which bond the polymers of a hydrogel fall under two general categories: physical and chemical. Chemical hydrogels have covalent cross-linking bonds, whereas physical hydrogels have non-covalent bonds. Chemical hydrogels result in strong irreversible gels due to the covalent bonding, and they may also possess harmful properties which makes them unfavorable for medical applications. Chemical crosslinks consist of covalent bonds between polymer strands. Hydrogels generated in this manner are sometimes called ‘permanent’ hydrogels.

Physical hydrogels on the other hand have high biocompatibility and are not toxic. Reversibility of physical hydrogels has been demonstrated by others only through changing an external stimulus such as pH or temperature. Physical crosslinks consist of hydrogen bonds, hydrophobic interactions, and chain entanglements (among others).

Hydrogels are prepared using a variety of polymeric materials, which can be divided broadly into two categories according to their origin: natural or synthetic polymers. Natural polymers for hydrogel preparation include hyaluronic acid, chitosan, heparin, alginate, and fibrin. Common synthetic polymers include polyvinyl alcohol, polyethylene glycol, sodium polyacrylate, acrylate polymers and copolymers thereof.

There are two suggested mechanisms behind physical hydrogel formation, the first one being the gelation of nanofibrous peptide assemblies, usually observed for oligopeptide precursors. The precursors self-assemble into fibers, tapes, tubes, or ribbons that entangle to form non-covalent cross-links. The second mechanism involves non-covalent interactions of crosslinked domains that are separated by water-soluble linkers, and this is usually observed in longer multi-domain structures. Tuning of the supramolecular interactions to produce a self-supporting network that does not precipitate, and is also able to immobilize water which is vital for to gel formation.

It is to be appreciated electroadhesion can also be induced between two physical gels of opposite charge, or between a physical and a chemical gel. In particular, millimeter-scale spherical capsules made from biopolymers (alginate, chitosan) by ionic crosslinking can be strongly adhered despite a small contact area. In such cases, electroadhesion can be induced rapidly (in 10-60 s) by low voltages (3-25 V DC) and is completely reversible. For example, adhesion can be achieved with voltages as low as 3V if time is extended for the gel-tissue or gel-gel in the field. The adhesion is strong enough to allow capsules/gels to be assembled in three dimensions (3D) into robust structures. Such 3D structures can include capsule-capsule chains, capsule arrays on a base gel, and a 3D cube. Electroadhesion-based assembly of spherical building blocks can be done faster and more easily than by any alternative techniques. Electroadhesion can also be used for selective sorting of charged soft matter—for example, a ‘finger robot’ can selectively “pick up” capsules of the opposite charge by electroadhesion and subsequently “drop off” these structures by reversing the polarity. Overall, electric fields can be used to conveniently manipulate a diverse range of soft matter.

### EXAMPLES

Embodiments of the present invention are further defined in the following non-limiting Examples. It should be understood that these Examples, while indicating certain embodiments of the invention, are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims.

### Gel-Gel Electroadhesion

Electroadhesion was used to repair punctures 108, cuts 112 or broken gel-tubes 100. A cut 112 in the wall of an alginate tube 100 can be repaired with a QDM gel 200+ placed over the cut. For purposes of the examples, the cut 112 can be made with a needle or razor blade and its size can be varied. Rectangular patches of the QDM gel 200+(e.g., 15 mm long, 8 mm wide and 2 mm in thickness) can be used in the electroadhesion procedure described above. The QDM gel patch 200+ was affixed so as to cover the cut 112 in the tube wall. Note that the patch adheres tightly to the tube 100 and conforms to the tube's curvature, as shown in FIG. 2B.

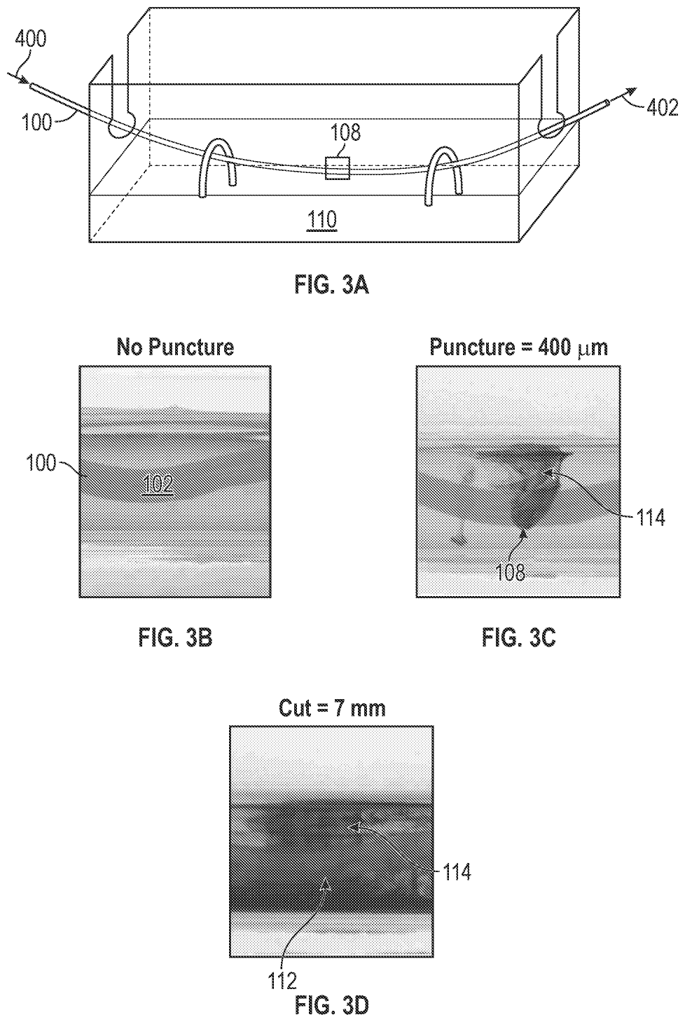

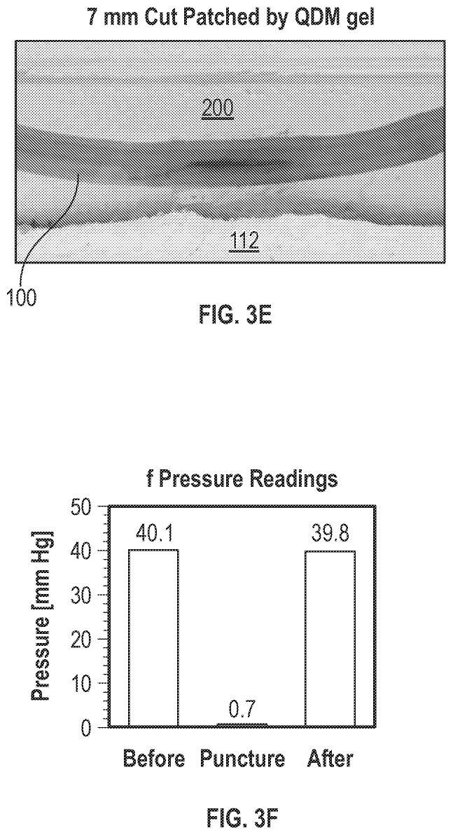

To test the strength of the patch-tube adhesion, flow 400 of fluid was introduced through the patched tube 100. If the patch 200 was not affixed, fluid would leak out of the cut 112 in the tube 100. Results from the test determined whether the patch could completely seal the leak and whether the patch could withstand the pressure exerted by the fluid. A protocol for leakage tests involves submerging the Alg tube 100 in a water bath 110 comprising 0.1% of tannic acid, as shown in FIGS. 3A-3B. A 0.1 wt % solution of iron chloride (FeCl₃) in water is then flowed through the lumen of the tube using a peristaltic pump. When FeCl₃ contacts tannic acid, a black precipitate of ferric tannate 114 is instantly formed. Even if a small puncture 108 (400 m) is made in the tube wall using a needle, the leakage of fluid from the puncture can be readily detected by the eye due to the formation of the black precipitate 114 (FIG. 3C). This leakage is much greater when a large cut 112 (7 mm in length) is made in the tube wall with a blade (FIG. 3D). FIG. 3E shows the alginate tube 100 with the above large cut patched by a QDM gel 200 using electroadhesion. In this case, there is stable flow 400 of fluid through the tube 100 with no leak whatsoever.

To quantify the above example, a pressure gauge was installed to the tube upstream of the puncture site. This measures the pressure P exerted by the fluid flow on the tube wall. When some of the fluid leaks out through the cut 112, P drops relative to its initial value (which corresponds to stable flow with no leak). If the leak is considerable, then P drops to nearly zero. For example, the bar graph in FIG. 3F shows that the initial P is 40.1 mm-Hg as fluid is flowed through the tube 100 at a flow rate of 5 mL/min. When a cut of 7 mm length is made in the tube wall and flow is resumed at the above flow rate, P drops to 0.7 mm-Hg. Next, the cut is patched with a QDM gel using electroadhesion and the example is repeated—P then increases to 39.8 mm-Hg, which is nearly the same as the initial pressure. The data shows that the electroadhered patch holds up well to the above flow conditions.

The pressure readings above depend on both the flow conditions and the size of the cut 112 in the tube 100. If the flow rate is increased above a critical value, the pressure exerted by the fluid is able to dislodge the electroadhered patch and the fluid then leaks into the surrounding bath 110. The pressure at this point of failure is defined as the burst pressure, “Pburst”. Pburst represents a limit for the conditions studied.

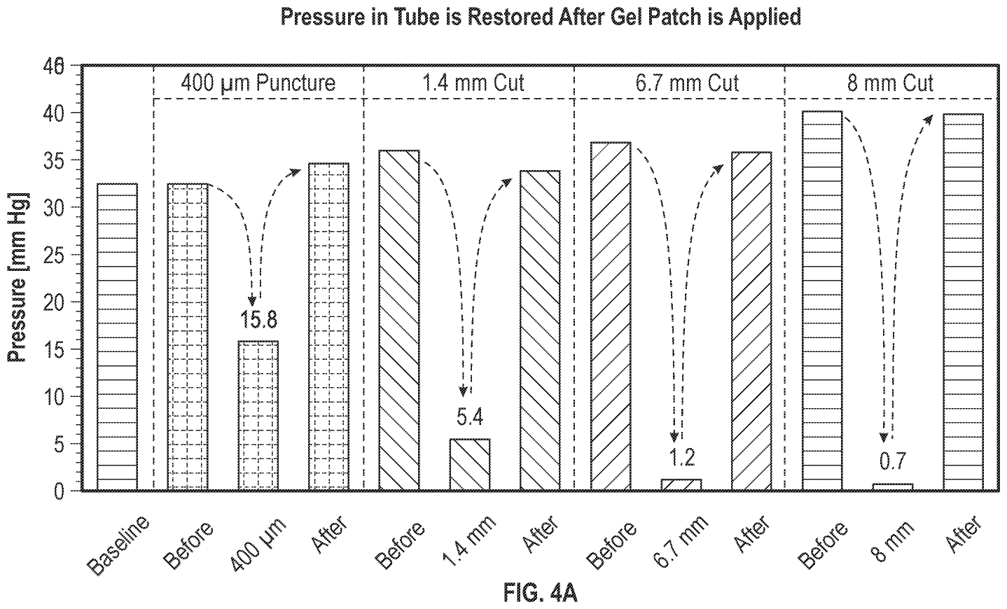

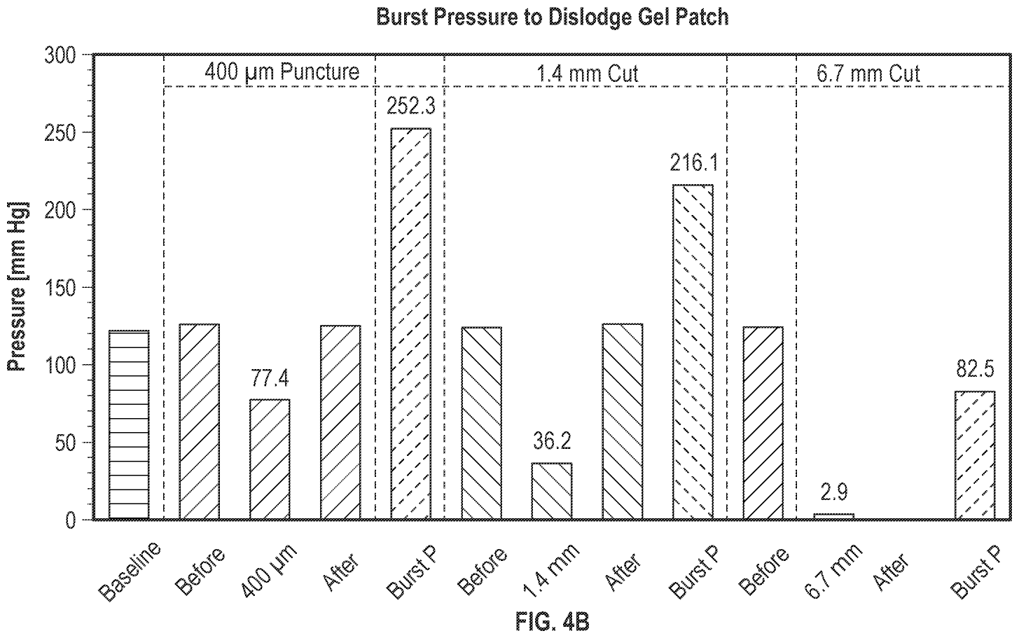

FIGS. 4A-4B shows pressure readings for various puncture/cut sizes. Pburst is 252 mm-Hg for a small (0.4 mm) puncture, 216 mm-Hg for a medium (1.4 mm) puncture, and 82 mm-Hg for a large (7 mm) cut. These Pburst values indicate robust sealing capability under typical blood-flow conditions (normal systolic blood pressure being 120 mm-Hg in healthy humans). Note that Pburst can be easily increased by either using a larger gel-patch around the cut or by introducing a second gel-patch over the first in a cross geometry.

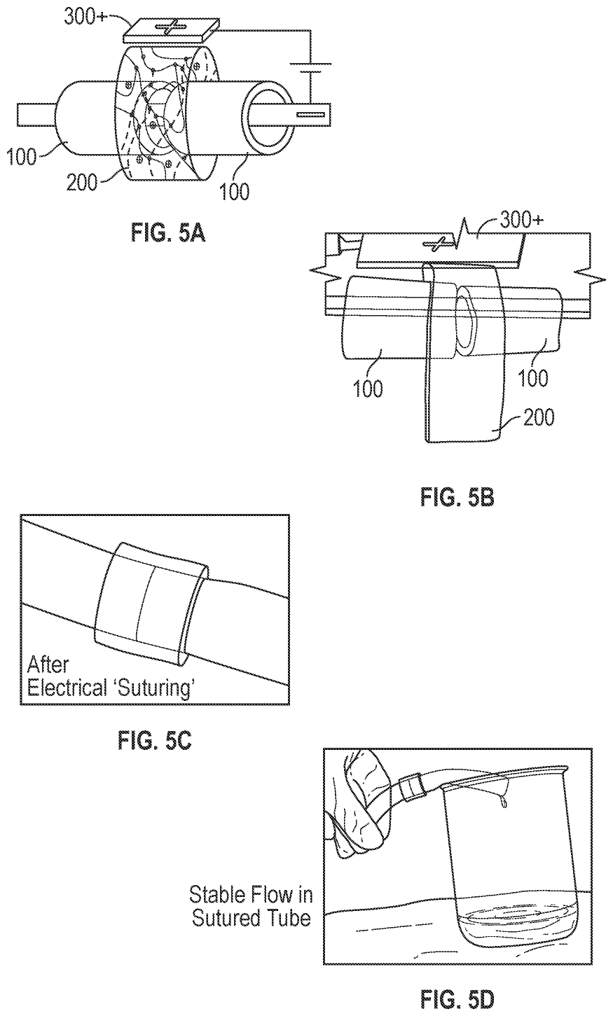

Electroadhesion can repair a much more extreme ‘injury’ compared to a cut in the tube wall. In this case, the alginate tube 100 was severed in half and attempted to join the two pieces using a QDM gel-strip 200. First, a long and flexible gel-strip 200 (15 mm long, 8 mm wide and 2.5 mm in thickness) was made. The two pieces of the tube were contacted laterally and the QDM gel-strip was wrapped around the tube segments, as shown in FIG. 5A. During electroadhesion, the negative electrode 300− was kept in contact with the tube 100− at all times while the positive electrode 300+ was rotated along the exterior of the gel-strip 200+, as shown in FIG. 5B.

The net result is that the QDM gel functions as a sleeve that wraps around the cut piece, as shown in FIG. 5C. Note that the length of the gel strip 200 was chosen to match the perimeter of the sleeve so that there is no gap between the ends of the strip 200. Thus, the patched tube 100&200 behaves like a single entity. Fluid can then flow 400 through the patched tube without leaks, as shown in FIG. 5D.

### Gel-Tissue Electroadhesion

Further, the electroadhesion of gels 200 can be extended to soft materials other than gels. There are many naturally occurring soft materials in nature. These include tissues of various creatures, including mammals, birds, worms, aquatic creatures as well as plant material and food products. For example, the gels 200 can be electroadhered to animal tissues 500 that form animal organs. Electroadhesion can apply to a broad spectrum of organisms and tissues, such as but not limited to: bovine (e.g., cows), porcine (e.g., pigs), murine (e.g., rodents such as mice), avine (e.g., birds such as chickens), piscine (e.g., fish such as salmon), crustaceans (e.g., shrimp, crab, etc.), and plants (e.g., strawberries, carrot, lettuce, etc.). Example organs that can electroadhere include, but are not limited to, aorta, cornea, intestines, lung, muscle, tendon, cartilage, fascia, and dermis.

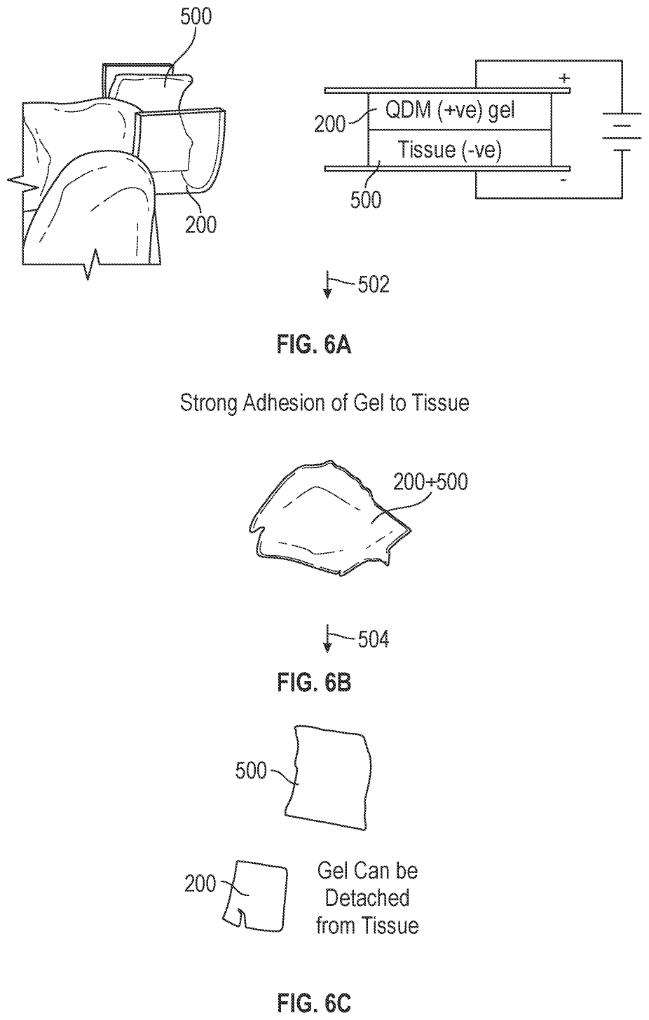

Bovine tissues from a bovine aorta were cleaned and prepared for the present example. The bovine tissue samples 500 were then tested along with the same QDM gels 200 as above, as shown in FIGS. 6A-6C. A piece of bovine aorta was first tested, which is one of the largest arteries in an animal. A rectangular strip 200 (1.5×2.5 cm²) of the aorta was used along with a similar strip of the QDM gel. As shown in FIG. 5A, the gel and tissue are contacted with electrodes 300 in the same orientation as before (E⁺G⁺T⁻E⁻), with the cationic QDM gel (G) connected to the positive electrode and the tissue (T) to the negative electrode. A direct current DC voltage of ten volts (10 V) is then applied for approximately twenty seconds (˜20 s), whereupon the gel becomes strongly adhered to the tissue, FIG. 5B. This suggests that the tissue behaves like an anionic gel, and hence the notation: T⁻.

No adhesion is observed if the reverse orientation (E⁺T⁻G⁺E⁻) of the field is used. Also, if the electroadhered gel-tissue pair is placed in the reverse orientation and the field is applied, the gel-tissue adhesion is reversed and the two can be easily separated, as in the gel-gel case, as shown in FIG. 5C. Therefore, the QDM gel can be reversibly electroadhered to the aorta.

Regarding the strength of electroadhesion between gel and tissue, it is to be appreciated that when the QDM gel is contacted with the aorta in the absence of the field, a weak adhesion is found, which is termed ‘contact adhesion’. The contact adhesion is weak enough that the gel can be peeled off intact from the tissue by hand without much force. In contrast, when the gel is electroadhered to the aorta, the gel cannot be peeled off intact by hand or lifted off from the aorta by using a scalpel. Thus, strong adhesion of the gel to the tissue is induced by the field and that this adhesion is much stronger than the contact adhesion between the two.

Once electroadhesion of the QDM gel to the aorta was confirmed, the same phenomenon can be observed with other classes of bovine tissue. In all cases, a strip of tissue was cut similar to that in FIGS. 6A-6C and tested it against a similar strip of QDM gel. First, it was determined whether there was ‘contact adhesion’ when the strips of gel and tissue were pressed together without a field. The extent of adhesion (or lack thereof) was assessed using a subjective scale for adhesion strength, as can be seen in Table 1. For this purpose, there was an attempt to detach the gel from the tissue and noted the ease with which this could be done.

The results were then classified into: 0=negligible, 1=weak, 2=moderate, 3=strong, and 4=very strong adhesion. For a given gel tissue pair, the results for ‘contact adhesion’ provided the baseline. Next, electroadhesion was induced of the same gel-tissue pair using the same protocol as in FIGS. 6A-6C (i.e., using 10 V of DC, applied for 20 s). After the field was switched off, the adhesion strength was assessed using the above 0-4 scale and compared the results to the baseline. The results for all types of tissue are presented in Table 1.

TABLE 1
Results of electroadhesion tests done with QDM gels and various bovine
tissues. Electroadhesion was significant relative to contact adhesion
only for the tissues listed in the left half of the table.

| Adhesion | | | No adhesion | | |
|---|---|---|---|---|---|
| | Adhesion | Adhesion | | Adhesion | Adhesion |
| | Strength | Strength | | Strength | Strength |
| | Following | Following | | Following | Following |
| Tissue | Electro- | Contact | Tissue | Electro- | Contact |
| (Bovine) | adhesion | Adhesion | (Bovine) | adhesion | Adhesion |
| Aorta | 3 | 1-2 | Heart | 0-1 | 0 |
| (descending | | | | | |
| thoracic) | | | | | |
| Cornea (inner | 3-4 | 0 | Brain | 0-1 | 0 |
| layer) | | | | | |
| Lung | 2 | 0 | Spleen | 0-1 | 0 |
| Cartilage | 2 | 0 | Fat | 0 | 0 |
| (articular) | | | | | |
| | | | Thymus | 1-2 | 1 |
| Tendon | 3-4 | 0 | Tendon | 1-2 | 0 |
| | | | (longitudinal | | |
| | | | section) | | |
| Skeletal | 2-3 | 0 | Skeletal | 1 | 0 |
| muscle (neck, | | | muscle (neck, | | |
| transverse | | | longitudinal | | |
| section) | | | section) | | |
| Skeletal | 2 | 0 | Skeletal | 1-2 | 0 |
| muscle (cheek, | | | muscle (cheek, | | |
| transverse | | | longitudinal | | |
| section) | | | section) | | |

The left half of Table 1 indicates several tissues for which the strength of electroadhesion is much higher than the baseline case of contact adhesion. The largest contrast is in the case of the cornea from the eye, where the QDM gel shows negligible contact adhesion (0 on the scale), but very strong electroadhesion (˜ 4 on the scale). Other tissues for which electroadhesion is clearly stronger and distinct relative to the baseline include the lung, the cartilage, and certain types of skeletal muscle. The case of the aorta, which was depicted above in FIGS. 6A-6C is one in which contact adhesion is not zero, but electroadhesion is clearly much stronger. Conversely, the right half of Table 1 lists the tissues for which electroadhesion is not significant under the conditions studied. In the case of the heart, the brain, the spleen, and fat tissue, there is no significant adhesion, either on contact or due to the field. In the case of the thymus, weak adhesion is observed due to the field, but this is not sufficiently distinguishable from contact adhesion. Tissues can be structurally complex, and the complexity is especially evident in our studies with tendon and skeletal muscle (bottom three entries in Table 1). If these tissues are cut in a longitudinal section, the samples do not exhibit significant electroadhesion; however if the same tissues are cut in a transverse section, electroadhesion is appreciable. Thus, there is significant anisotropy in the tissue structure, which also affects the results here. All in all, it can be concluded from Table 1 that cationic QDM gels can be electroadhered to some types of animal tissue.

Incidentally, with regard to the voltage, electroadhesion of gels to tissues can be achieved even with voltages as low as 3 V but applied for a longer duration (˜60-120 s). No adhesion was seen for voltages below 3 V for both gel-tissue and gel-gel systems, largely consistent with previous studies. Conversely, with some of the tissues in Table 1 for which electroadhesion was unsuccessful with the current protocol (10 V for 20 s), a longer. It is to be appreciated increasing application time (e.g., for 60 s) could potentially enable strong electroadhesion with more of the tissue types listed in Table 1.

Electroadhesion work with some types of tissues and not others. Anionic counterparts to the QDM gel 200 were made by copolymerizing AAm with an anionic monomer like sodium acrylate (SA). However, this gel could not be electroadhered to any tissues. Thus, in all the successful cases of electroadhesion, the gel 200 was cationic (i.e., QDM), which implies that the tissue 500 must be anionic. Animal tissues 500 are expected to have a microstructure consisting of cells (either discrete or close-packed into clusters) embedded in a network of polymer chains, i.e., the extracellular matrix (ECM). The ECM tends to have different composition in different tissues. Two key proteins in the ECM are collagen and elastin. The percentage of each of these proteins in the tissues is indicated below, if it were found.

These numbers are shown in Table 2, which is divided into two halves similar to Table 1, with the tissues exhibiting electro-adhesion on the left and those that do not on the right. In addition to the proteins, the water content in each tissue is also shown.

TABLE 2
Collagen, elastin, and water content of tissues
that do and do not exhibit electroadhesion.

| Adhesion | | | | No adhesion | | | |
|---|---|---|---|---|---|---|---|
| Tissue | Percent | Percent | Percent | Tissue | Percent | Percent | Percent |
| (Bovine*) | Collagen | Elastin | Water | (Bovine*) | Collagen | Elastin | Water |
| Aorta | 23 | 40 | 84-87 | Heart | 3 | 0 | 74 |
| Cornea | 70 | | 78 | Brain | | 0 | 73 |
| Lung | 11 | 5 | 84 | Spleen | 0-1 | 5 | 79 |
| Cartilage | 58 | | 80 | Fat | 0 | 9 | 50 |
| | | | | Thymus | 1-2 | | |
| Tendon | 85 | 5 | 55-70 | Tendon | 1-2 | 5 | 55-70 |
| (transverse | | | | (longitudinal | | | |
| section) | | | | section) | | | |
| Skeletal | 2-7 | 0 | 80 | Skeletal | 1 | 0 | 80 |
| muscle | | | | muscle | | | |
| (neck, | | | | (longitudinal | | | |
| transverse | | | | section) | | | |
| section) | | | | | | | |

*Values correspond to bovine tissue unless otherwise noted below. Values are rounded to the nearest integer.
Notes:
a. Aorta: Collagen and elastin compositions of aorta, water content.
b. Cornea: Collagen composition, elastin percent values not given, water content.
c. Lung: Collagen and elastin compositions of rat lung, water content
d. Cartilage: Collagen composition of human cartilage, elastin percent values not given, water content.
e. Tendon: Collagen and elastin compositions of tendon, water content.
f. Skeletal muscle: Collagen and elastin compositions of skeletal muscle, water content.
g. Heart: Collagen and elastin compositions of heart, water content.
h. Brain: Collagen and elastin compositions of rat brain, water content.
i. Spleen: Collagen and elastin compositions of spleen, water content.
j. Fat: Collagen percent values not given, elastin composition of human adipose (fat) tissue, water content.
k. Thymus: Collagen percent values not given, no data on elastin and water content available in literature.

One observation from Table 2 is that many (but not all) the tissues in the left half have a high collagen content. Collagen itself is a protein that has a net neutral charge at ambient pH, and on its own would not impart anionic character to the tissue. However, collagen-rich tissues often are associated with protein-sugar hybrid polymers called glycosaminoglycans (GAGs), which are known to be strongly anionic. The GAGs anchor cells to the ECM by attaching simultaneously to proteins on the cell surface as well as to the collagen fibers in the ECM. GAGs such as heparan sulfate have high affinity to collagen type I and III, which are the main types of collagen in the aorta. Another observation from Table 2 is that some types of collagen-rich ECMs also have high concentrations of elastin. Elastin is reported to be cationic at ambient pH, which allows it to bind to GAGs via electrostatic interactions. Collectively, in a tissue that contains collagen, GAGs, elastin, and other polymers, the overall composition of charged polymers will dictate the net charge of the tissue. Tissues with a net anionic character have a propensity to undergo electroadhesion (to cationic gels like QDM). If the water content in the tissue is too low (such as in the case of fat or brain tissue), the tissue may not exhibit electroadhesion.

An additional factor to consider is the ionic strength of the (fluid in the) tissue. Interactions between cationic and anionic polymers will be impacted by the ionic strength. In this regard, the QDM gel and a representative tissue (aorta) can therefore be soaked in different fluids of biological relevance and then examined their adhesion. These fluids are expected to have an ionic strength around 0.15 M. If soaked in whole blood (bovine), the gel and tissue electroadhere just as in their native state. When soaked in blood plasma (bovine) or in phosphate-buffered saline (PBS), the gel-tissue adhesion was initially weaker, but built up thereafter. By increasing the time over which the field was applied from 20 s to 60 s, significant adhesion between the gel and tissue was obtained in all cases.

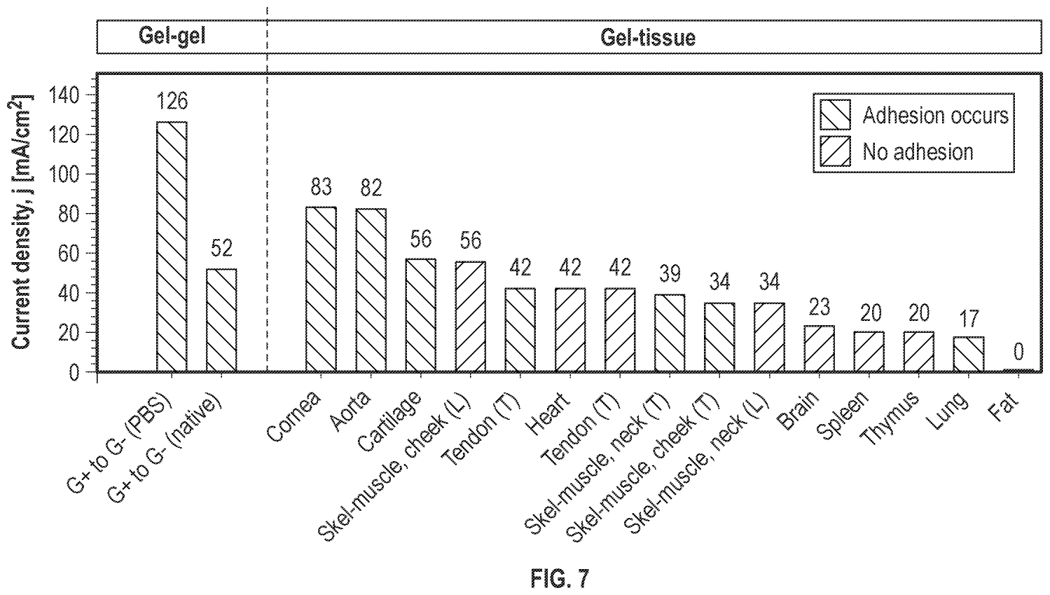

The current I during electroadhesion examples was recorded in FIG. 7, which is reported for various pairs. Data in FIG. 7 is shown for the current density j (i.e., I/area of contact). j seems to depend mainly on the ionic strength of the gel and tissue: for example, j is 52 mA/cm² when two gels (G⁺ and G⁻) are electroadhered in their native state (i.e., after preparation with deionized water) but increases to 126 mA/cm² when the same gels are soaked in PBS. Similar currents were observed even if gels of the same charge (e.g., two G^{T}) were contacted, which would be a case of no adhesion. In the case of gel-tissue examples, j for various tissues are shown in FIG. 7. For tissues that electroadhere, j varies from a low of 17 mA/cm² for the lung to around 80 mA/cm² for the aorta and cornea. For tissues that do not electroadhere, j is nearly zero for fat tissue, whereas it is 42 mA/cm² for heart tissue. From these numbers, no clear relationship can be discerned between j and adhesion (or lack thereof). It should be mentioned that the j values reported correspond to the highest current recorded, which is near the start of the example. With time, the current drops to a steady-state that is 20-30% of the above values.

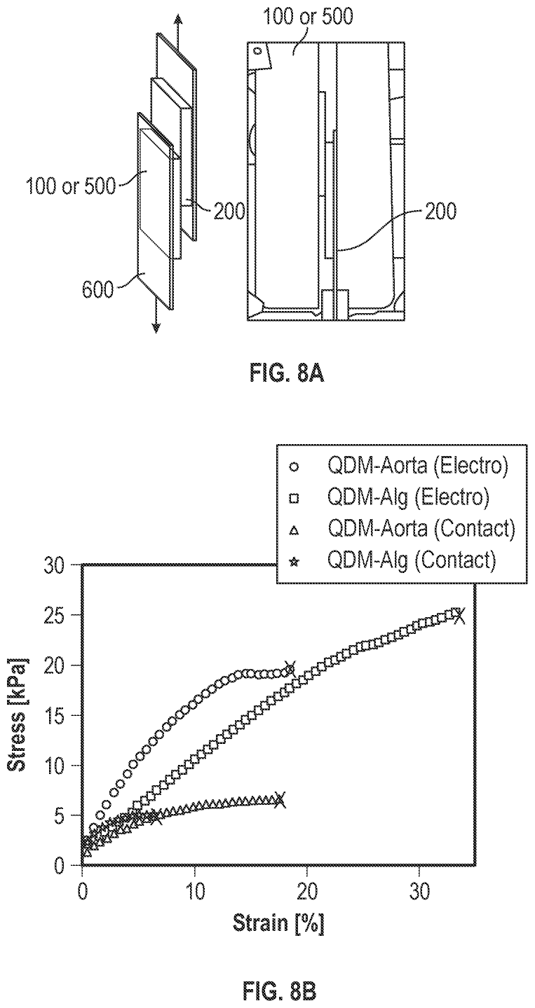

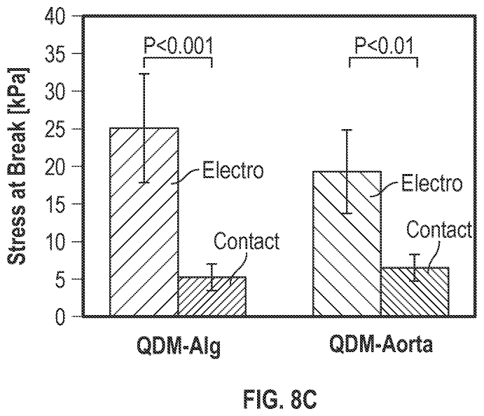

The gel-tissue adhesion strength was measured and compared to that for the gel-gel case. The measurements were done using the lap-shear test protocol, which is described in more detail in the Methods section below. In this test, two rectangular samples are adhered to each other over a portion of their area, which is called the ‘lap’, as shown in FIG. 8A. The outer surfaces of the two samples are then stuck to glass slides 600 using cyanoacrylate glue. The setup is then placed in the testing instrument, with each glass slide being gripped on its end by the jaws of the instrument. A tensile strain is then applied until failure occurs, and the magnitude of the stress-at-break is a measure of the adhesion strength. Stress vs. strain curves are presented in FIG. 8B for two sets of samples: a QDM gel adhered to an Alg gel, and the same QDM gel adhered to bovine aorta. For both cases, the test was run first under ‘contact adhesion’, where the samples are pressed together without a field. Next, the two samples are electroadhered and the test is repeated. In both the gel-gel and the gel-tissue cases, the stress-strain curves for electroadhesion extend up to much higher stresses compared to contact adhesion (FIG. 8B), indicating the strong adhesion imparted by the electric field.

The adhesion strengths determined from the above curves are plotted in FIG. 8C. The strength of gel-gel (QDM-Alg) electroadhesion is found to be approximately twenty five kilopascals (˜25 kPa). For comparison, previous attempts have measured the adhesion strength (using the same lap-shear technique) for a pair of cationic and anionic acrylamide-based gels and reported values of around only ten kilopascals (10 kPa). For the electroadhered gel-tissue pair (QDMaorta), the adhesion strength is about twenty kilopascals (20 kPa), which is comparable to that for the gel-gel case. In both cases, the strength of contact adhesion is much lower (˜ 5 kPa). These measurements confirm that, for both the gel-gel and gel-tissue cases, electroadhesion is substantially strong.

In the gel-gel case, when failure occurred, it was generally a cohesive failure, i.e., pieces of each gel were found to remain on the other. In the gel-tissue case, failure was also generally cohesive. Whereas some gel remained stuck to the tissue, no tissue remained stuck to the gel. This difference could be because the tissue tested (aorta) was generally much stiffer than the QDM and Alg gels.

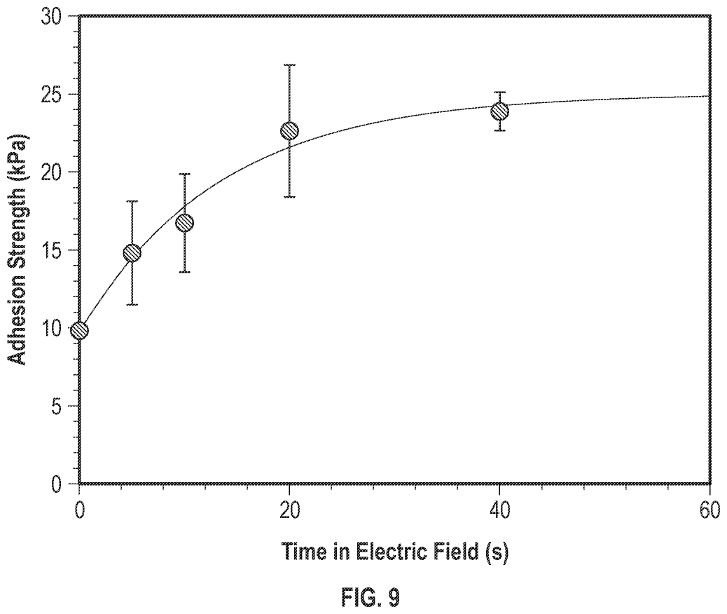

The adhesion strength is a function of time under the electric field, and the corresponding data shows this by way of FIG. 9. These data are for QDM gels crosslinked with BIS (but not containing LAP nanoparticles) in contact with bovine aorta. The same lap-shear protocol as in FIGS. 8A-8C was used and the stress-at-break was used as a measure of adhesion strength. Gel-tissue pairs were placed for different times in an electric field generated by ten volts direct current (10 V DC). The data reveal that sufficient electroadhesion (i.e., much higher than contact adhesion) develops within ten seconds (10 s) in the field.

Subsequently, the adhesion strength tapers off to a constant value by about twenty seconds (20 s), and similar values are obtained with higher contact times (e.g., forty seconds: 40 s). Thus, a time of twenty seconds (20 s) in the field seems to be more than adequate to induce appreciable electroadhesion between gel and tissue. Similar data for adhesion strength as a function of contact time has been reported previously for gel-gel adhesion.

### Electroadhesion for Repairing Cut or Damaged Tissue

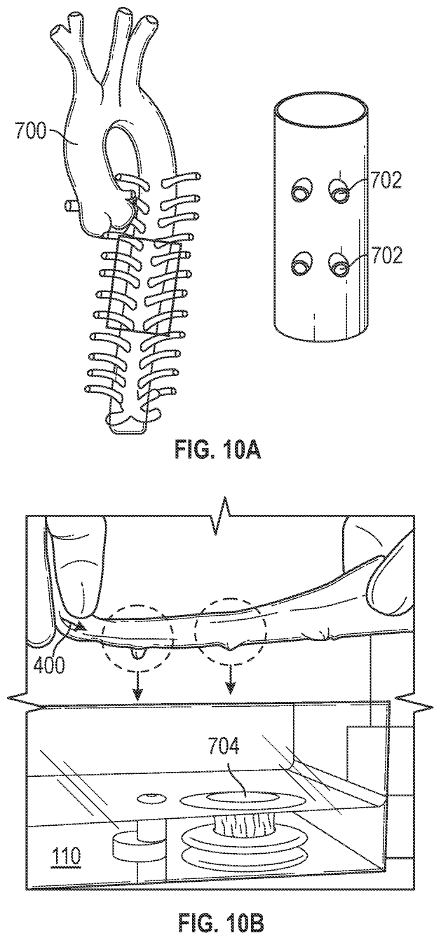

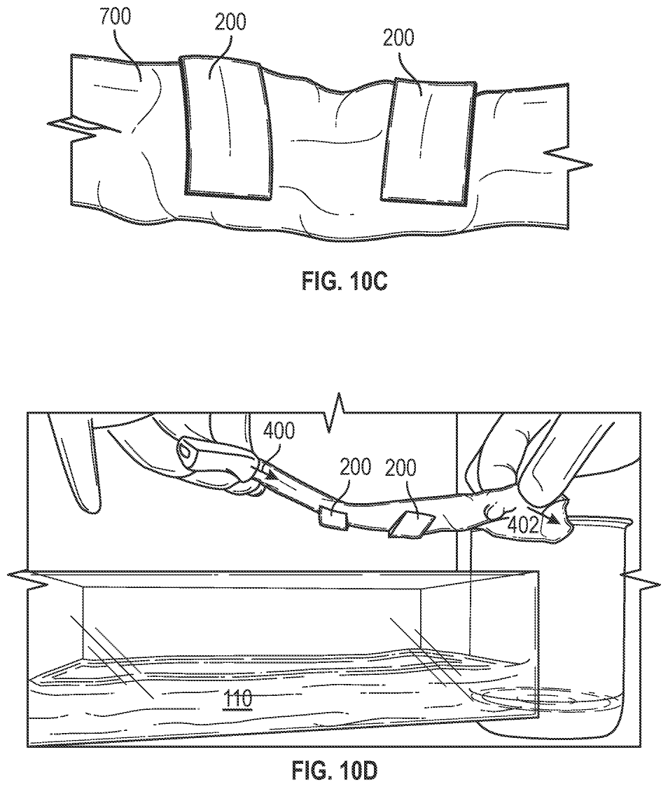

An electroadhered gel patch can seal cuts on a tissue, effectively mimicking a surgical repair. These examples are similar to those previously demonstrated above with the anionic gel tube in FIGS. 3A-F and FIGS. 5A-5D, where cuts 112 were sealed by a QDM gel patch 200. The example related to FIG. 9 again employed the cationic QDM gel 200, but this time the example involved a segment from the descending thoracic aorta 700 of a cow, which was about 15 cm long and 2 to 2.5 cm in diameter (FIG. 10A). The aorta 700 has pairs of holes along its length which correspond to arterioles 702. Arterioles 702 are small branches from the aorta 700 that transport blood to various organs. If the aorta is used as a tube, fluid will leak out through the arterioles. This is illustrated by FIG. 10B, which employed similar test protocol to that of FIGS. 3A-F. A solution of 0.1% FeCl3 is pumped through the lumen of the aorta 700. The fluid leaks out through the arterioles 702 and drips into the bath 110 containing 0.1% tannic acid, whereupon a black precipitate 114 of ferric tannate is instantly formed. Note that the aorta 700 was not submerged in the bath to avoid any reaction of the tissue with the tannic acid.

Next, two rectangular patches of the QDM gel 200 were made for the two pairs of arterioles 702 in the aorta 700. The gel patches 200 were affixed over the arterioles 702 (one patch covers two adjacent arterioles) using electroadhesion (10 V, 20 s). The gels adhered tightly to the tissue, as can be seen in FIG. 10C. Thereafter, the flow of FeCl₃ solution through the aorta was restarted. FIG. 10D shows there are no leaks through the arterioles 702, i.e., the holes remain sealed, allowing fluid to flow right through the aorta 700. The output fluid 402 was collected in a beaker containing 0.1% tannic acid at the end of the aorta 700. The black precipitate 114 of ferric tannate is seen in the beaker but not in the water bath 110, thus confirming that there are no leaks through the tissue during the flow process.

### Methods

Materials. The following chemicals were from Sigma-Aldrich: the monomers acrylamide (AAm) and N,N′-methylenebis(acrylamide) (BIS), the initiator ammonium persulfate (APS), calcium chloride dihydrate (CaCl₂)) salt, tannic acid, sodium hydroxide, phosphate buffered saline (PBS) tablets, and the dye rhodamine B. The accelerant N,N,N′,N′-tetramethylethylene-diamine (TEMED) was from TCI America. The monomer N,N′-dimethylaminoethyl methacrylate, quatemary ammonium salt (QDM) was from MPD Chemicals. Two biopolymers were purchased from Sigma-Aldrich: alginate (Alg) (from brown algae, medium viscosity) and agarose (Type 1-A, low EEO, melting temperature ˜88° C.). Laponite XLG nanoparticles (LAP) were a gift from Southern Clay Products. Cyanoacrylate-based glues (Gorilla Glue gel and Krazy Glue) and Rust-Oleum hydrophobic coating were purchased from The Home Depot. Deionized (DI) water was used in the examples described herein.

Synthesis of Alginate Tubes. Alginate tubes were prepared by first using a template of cylindrical agarose gel containing Ca²⁺ ions was prepared. For this, 2.5 wt % of agarose and 5 wt % of CaCl₂) were added to DI water and heated above 80° C. until the agarose completely dissolved. The hot solution was then poured into a tube that was capped at one end. Upon cooling to room temperature, a solid (gel) cylinder of agarose was obtained. This cylinder was then placed in a solution of 2 wt % Alg for 12 min. During this time, Ca²⁺ ions diffuse out of the agarose, leading to an Alg gel around the cylindrical core. The final step was to dip this material in a 3 wt % CaCl₂) solution for 20 min and then cut off the edges. The tube of Alg could then be slid off the agarose core. Alg tubes can be prepared over a range of dimensions using this method. The tubes were prepared in two typical dimensions by using agarose cores of different diameters and lengths: (a) 1 cm diameter and 10 cm length; and (b) 2 mm diameter and 60 cm length. Tubes were stored in a 1 wt % CaCl₂) solution and dyed with 0.1 mM rhodamine B for contrast purposes. Typically, tubeswere used within 24 h of preparation.

Synthesis of QDM gels. Cationic QDM gels were prepared using the following protocol. First, DI water was degassed by bubbling nitrogen gas for 30 min. To assist with easy removal of the gels, Petri dishes used in gel preparation were coated with a spray of Rust-Oleum hydrophobic coating, then allowed to sit for 10 min, and thereafter wiped dry. Two variations of QDM gels were prepared: with and without LAP. For synthesis of QDM gel without LAP the following were combined 1 M (1.4 g) Aam, 0.16 M (809 μL) of QDM solution, 0.019 M (0.06 g) BIS, 0.0088 M (0.04 g) APS and 0.01 M (30 μL) TEMED in 20 mL of degassed DI water. Next, the above monomer mixture was poured into a pre-coated Petri dish and maintained in a nitrogen environment for 3 h, whereupon the gel became fully polymerized. For synthesis of QDM gel with LAP, the first step was to add 1 wt % (0.2 g) of LAP particles to 20 mL degassed water and to stir until the particles were well-suspended (as ascertained by the sample appearing clear and homogeneous).

Thereafter, the pH of the solution was lowered to 4.5 using 1 M HCl. Next, 0.16 M (809 μL) QDM was added dropwise to the LAP mixture followed by 1 M (1.4 g) AAm, 0.0095 M (0.03 g) BIS, 0.0088 M (0.04 g) APS and 0.01 M (30 μL) TEMED. When the pH was below 5, QDM was able to dissolve in the LAP suspension (without clumping). TEMED increased the pH back up to around 8.5. The above solution was placed in a pre-coated Petri dish and polymerized as before. After polymerization, gels were stored in a fridge and typically used within 24 h of preparation.

Synthesis of SA gels. Anionic SA gels were prepared by a similar procedure as described above for QDM gels. In this case, the monomer solution contained 1.4 M (2 g) AAm, 0.11 M (0.2 g) SA, 0.019 M (0.06 g) BIS, 0.0088M (0.04 g) APS and 0.01 M (30 μL) TEMED in 20 mL of degassed DI water. The above solution was poured into a pre-coated Petri dish and maintained in a nitrogen environment for 2 h. After polymerization, gels were stored in a fridge and typically used within 24 h of preparation.

Tissue Preparation Protocol. All tissues were obtained ethically, immediately after slaughter from a local butcher. All experiments on tissues were conducted within 24 h of tissue harvest. When the tissues were first received, organs were typically encased in fat and other matrix material. For example, the aorta was surrounded with fat and connected to parts of the heart and lungs. Thus, for experiments with the aorta, it had to be harvested and cleaned from the surrounding parts. The harvested aorta was then further segmented into smaller pieces for the electroadhesion experiments, as shown in the above figure. For many experiments, segments of tissue were sliced to a thickness of 0.3±0.1 mm. The exceptions were in the cases of tissues that were naturally thin, such as the cornea.

Adhesion Experiments. A DC power source (Agilent, model E3612A) with a range of 0-60 V, 0-0.5 A was used for the electroadhesion experiments. The voltage was set to 10 V for most experiments. Graphite electrodes (from Saturn Industries) were cut to a size of 2×3×0.15 cm, and these were connected to the DC power source using alligator clips. The electrodes were placed on either side of the gel-gel pair or gel-tissue pair, as shown in FIGS. 2A-B and FIGS. 6A-6C. The gel strips were generally 2 mm in thickness, while the tissue strips were between 2 to 5 mm in thickness. Thus, the electric field strength across the gel-tissue sandwich was between 1.4-2.5 V/mm. For the gel-tissue experiments reported in Table 1, the following procedure was used. From a given batch obtained from the butcher, tissues of interest were harvested, and for a given tissue type, at least three tissue samples were prepared as described above. Three QDM gel strips were then prepared. First gel-tissue contact adhesion was measured, and then their electroadhesion. Two observers were used to independently rank the adhesion strength in each experiment on a scale of 0-4, where 0=negligible, 1=weak, 2=moderate, 3=strong, and 4=very strong adhesion (this scale is also indicated at the bottom of Table 1). The average of both observers' rankings was recorded for that experiment. Whenever in question, the second observer was blinded to the sample type so that their assessment was not biased. After three such trials with a tissue, the average of the readings was determined, and this is the one shown in Table 1.

Pressure Testing. The test setup is shown schematically in FIG. 6A. A peristaltic pump (Pharmacia-LKB-pump P-1) was used to pump a 0.1% FeCl3 solution through the Alg tube at a flow rate 5 mL/min. The tube was placed in a basin with a length of 15 cm, width of 5 cm and height of 5 cm. Openings were made on both sides to allow passage of the tube. The basin was filled with 0.1% tannic acid solution up to a height of 2 cm and the Alg tube (60 cm in length) was placed such that its middle portion was submerged in this solution (see FIG. 6A). Clamps were fixed at the bottom of the basin to control the path and location of the Alg tube in the basin. A pressure gauge (PRTemp 1000, from Madge Tech) was placed upstream of the Alg tube and the pressure was recorded in real-time (every 2 s) on a computer using the Madge Tech software. Pressure readings were obtained during flow in the tube before puncture, during puncture and following puncture repair by electroadhesion of a QDM gel patch. Burst pressures (for a patched tube) were determined by clamping shut the far end of the Alg tube and continuing flow into the tube, leading to pressure build up within the tube. The highest pressure recorded before the patch became dislodged was designated as the burst pressure. All measurements correspond to individual trials.

Lap-Shear Testing. Lap-shear tests were conducted using an Instron Model 5565 instrument. Tests were done according to protocols recommended by the American Society for Testing and Materials (ASTM) which have been used in previous studies. 33-35 Gels and tissues were cut into rectangular segments with dimensions of 1.5×4 cm. The QDM gel segment was 3 mm thick, the Alg segment was 1 mm thick and tissue segments were 2.5±1 mm thick. Gel-gel and gel-tissue samples were electroadhered over a lap height of ˜ 1.5 cm (see FIG. 8A). Following electroadhesion, the dangling ends of the gel and tissue were stuck securely to glass slides using cyanoacrylate glue. For securing the QDM and Alg gels to the glass slides, Krazy Glue was found to be the best and a 1 h cure time was used. For securing tissue to the same slides, Gorilla Glue was the best and a 2 h cure time was used (during this time, the tissue face exposed to air was covered by a piece of gauze soaked with PBS solution). The glass slides provided a hard and non-elastic backbone for the Instron to grip onto, which ensured that shear was applied on the lap area alone. The Instron was then used to elongate the sample at a rate of 10 mm/min, and the force was recorded during this process. At least three samples were tested for each of the categories in FIG. 8C, and the statistics were analyzed using the Student's T-test.

From the foregoing, it can be seen that the present disclosure accomplishes at least all of the stated objectives. For example, it has been demonstrated that electroadhesion can be applied to new materials and geometries. Cationic (QDM) gels and animal (bovine) tissues can be utilized for such electroadhesion. Gel and tissue were brought into contact with each other and with electrodes in a E⁺G⁺T⁻E⁻ orientation (i.e., with the cationic gel G⁺ touching the positive electrode E+ and the tissue T⁻ the negative electrode E⁻). A DC voltage of 10 V was then applied for 20 s, whereupon the gel became strongly adhered to the tissue, with the adhesion persisting after the field was turned off. The strength of adhesion between QDM gel and bovine aorta, measured by lap-shear testing, was ˜ 20 kPa. In addition to the aorta, electroadhesion also worked with the cornea, the lung, cartilage, and certain types of skeletal muscle and tendon. Cationic gels electroadhered to tissues, which implied that the tissues had anionic character. If the electroadhered gel-tissue pair was placed in a field with reversed polarity, the adhesion was lost and the two could be separated.

Additionally, electroadhesion can seal cuts or tears in tubes. Initial experiments in this regard were done with tubes of anionic Alg gel as a model system. As an extreme case, two severed pieces of an Alg tube were joined using an electroadhered QDM gel strip that was flexible enough to encircle the tube while spanning the cut segments. In a similar manner, in the case of bovine aorta, QDM gels were electroadhered over openings in the tissue (corresponding to arterioles). In both cases, the electroadhered patches provided a robust and durable seal, allowing fluid to flow right through the lumen of the tubes. The present disclosure raises the possibility of using electroadhesion to perform surgical repairs in the future. The use of strongly adhered gel patches could obviate the need for sutures or staples in many surgical procedures. The ability to achieve adhesion on command with an electric field, and moreover the ability to reverse the adhesion in case of an error, will enable surgeries to be done in a more rapid, durable, and precise manner.

### LIST OF REFERENCE CHARACTERS

The following table of reference characters and descriptors are not exhaustive, nor limiting, and include reasonable equivalents. If possible, elements identified by a reference character below and/or those elements which are near ubiquitous within the art can replace or supplement any element identified by another reference character.

TABLE 3
List of Reference Characters

| | |
|---|---|
| 100 | gel tube |
| 100− | anionic gel tube |
| 102 | diameter |
| 104 | Alg chains |
| 106 | Ca²⁺ ions |
| 108 | puncture site |
| 110 | bath (e.g. water bath containing tannic acid) |
| 112 | cut |
| 114 | black precipitate of ferric tannate |
| 200 | gel strip |
| 200+ | cationic gel strip |
| 202 | thickness |
| 204 | mixture comprising nonionic monomer, a cationic monomer, a |
| | nonionic crosslinker, and laponite (LAP) nanoparticles |
| 300 | electrodes |
| 300+ | positive electrode (anode) |
| 300− | negative electrode (cathode) |
| 400 | flow of iron chloride (FeCl₃) |
| 402 | output to pressure gauge |
| 500 | tissue |
| 502 | step for placing gel and tissue in direct current (DC) electric field |
| 504 | step for reversing adherence of gel and tissue by placing gel and |
| | tissue in direct current (DC) electric field with reversed polarity |
| 600 | glass slides |
| 700 | bovine thoracic aorta |
| 702 | arterioles |

### Glossary

Unless defined otherwise, all technical and scientific terms used above have the same meaning as commonly understood by one of ordinary skill in the art to which embodiments of the present disclosure pertain.

The terms “a,” “an,” and “the” include both singular and plural referents.

The term “or” is synonymous with “and/or” and means any one member or combination of members of a particular list.

As used herein, the term “exemplary” refers to an example, an instance, or an illustration, and does not indicate a most preferred embodiment unless otherwise stated.

The term “about” as used herein refer to slight variations in numerical quantities with respect to any quantifiable variable. Inadvertent error can occur, for example, through use of typical measuring techniques or equipment or from differences in the manufacture, source, or purity of components.

The term “substantially” refers to a great or significant extent. “Substantially” can thus refer to a plurality, majority, and/or a supermajority of said quantifiable variable, given proper context.

The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

The “invention” is not intended to refer to any single embodiment of the particular invention but encompass all possible embodiments as described in the specification and the claims. The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, subcombinations, or the like that would be obvious to those skilled in the art.

The present disclosure is further defined by the following numbered paragraphs:
1. A method comprising electroadhering a cationic hydrogel to anionic cells.
2. The method according to paragraph 1 further comprising: contacting a tissue comprised of said anionic cells with a negative electrode; contacting a cationic hydrogel with a positive electrode; bringing the cationic hydrogel and the tissue in contact within an electric field powered by direct current (DC) or alternating current (AC) for a finite period of time; and allowing the cationic hydrogel to electroadhere to the tissue.
3. The method according to paragraph 2 further comprising maintaining adhesion after the finite period of time has elapsed and the direct current (DC) or alternating current (AC) is no longer applied to either the positive electrode or the negative electrode.
4. The method according to any one of paragraphs 2-3 further comprising joining two groups of anionic cells with the cationic hydrogel.
5. The method according to any one of paragraphs 2-4 further comprising removing the adhesion between the anionic cells and the cationic hydrogel by applying an electric field of a reversed polarity for an additional finite period of time.
6. The method according to any one of paragraphs 2-5 wherein the group of anionic cells comprise collagen and elastin.
7. The method according to any one of paragraphs 2-6 further comprising patching a puncture or cut in the tissue.
8. The method according to any one of paragraphs 2-7 wherein the electroadhesion between the cationic hydrogel and the tissue is of a chemical type and includes: a. s-IPNs with a cationic charge; or b. monomers or co-monomers with a cationic charge.
9. The method according to any one of paragraphs 2-8 wherein the electroadhesion between the cationic hydrogel and the tissue is of a physical type.
10. The method according to any one of paragraphs 2-9 wherein the electroadhesion between the cationic hydrogel occurs in a double network wherein one or both networks has a cationic charge.
11. The method according to any one of paragraphs 2-10 further comprising:
flowing an iron chloride solution through the anionic cells; and
submerging the anionic cells in a water bath containing 0.1% of tannic acid to determine whether there are any leaks in the anionic cells.
12. The method according to any one of paragraphs 2-11 further comprising sealing arterioles using the electroadhered cationic hydrogel.
13. The method according to any one of paragraphs 2-12 further comprising measuring the gel-tissue adhesion strength between the anionic hydrogel and/or group of anionic cells and the cationic hydrogel by sticking samples onto glass slides using a cyanoacrylate glue.
14. A system for accomplishing sutureless tissue repair comprising: a cationic hydrogel; anionic cells; and electrodes adapted to (1) receive power from a direct current (DC) power supply; and (2) contact said cationic hydrogel and said anionic cells.
15. The system according to paragraph 14 wherein the anionic hydrogel is formed into a cylindrical tube.
16. The system according to any one of paragraphs 14-15 wherein the cationic hydrogel is formed into a strip.
17. An electroadhered material comprising: a covalently crosslinked gel electroadhered to a physically crosslinked gel.
18. The electroadhered material according to paragraph 17 wherein the covalently crosslinked gel comprises alginate crosslinked by divalent Ca²⁺ cations.
19. The electroadhered material according to any one of paragraphs 17-18 wherein the physically crosslinked gel comprises a mixture that includes a nonionic monomer, a cationic monomer, a nonionic crosslinker, and crystalline nanoparticles having a highly ionic surface area.
20. The electroadhered material according to paragraph 19 wherein the nonionic monomer comprises acrylamide.
21. The electroadhered material according to any one of paragraphs 19-20 wherein the cationic monomer comprises quaternized dimethyl aminoethyl methacrylate.
22. The electroadhered material according to any one of paragraphs 19-21 wherein the nonionic crosslinker comprises bis(acrylamide).
23. The electroadhered material according to any one of paragraphs 19-22 wherein the crystalline nanoparticles with the ionic surface area comprise laponite nanoparticles.

What is claimed is:

1. A method comprising electroadhering a cationic hydrogel to anionic cells.

2. The method of claim 1 further comprising:
contacting a tissue comprised of said anionic cells with a negative electrode;
contacting a cationic hydrogel with a positive electrode;
bringing the cationic hydrogel and the tissue in contact within an electric field powered by direct current (DC) or alternating current (AC) for a finite period of time; and
allowing the cationic hydrogel to electroadhere to the tissue.

3. The method of claim 2 further comprising maintaining adhesion after the finite period of time has elapsed and the direct current (DC) or alternating current (AC) is no longer applied to either the positive electrode or the negative electrode.

4. The method of claim 2 further comprising joining two groups of anionic cells with the cationic hydrogel.

5. The method of claim 2 further comprising removing the adhesion between the anionic cells and the cationic hydrogel by applying an electric field of a reversed polarity for an additional finite period of time.

6. The method of claim 2 wherein the group of anionic cells comprise collagen and elastin.

7. The method of claim 2 further comprising patching a puncture or cut in the tissue.

8. The method of claim 2 wherein the electroadhesion between the cationic hydrogel and the tissue is of a chemical type and includes:
a. s-IPNs with a cationic charge; or
b. monomers or co-monomers with a cationic charge.

9. The method of claim 2 wherein the electroadhesion between the cationic hydrogel and the tissue is of a physical type.

10. The method of claim 2 wherein the electroadhesion between the cationic hydrogel occurs in a double network wherein one or both networks has a cationic charge.

11. The method of claim 2 further comprising:
flowing an iron chloride solution through the anionic cells; and
submerging the anionic cells in a water bath containing 0.10% of tannic acid to determine whether there are any leaks in the anionic cells.

12. The method of claim 2 further comprising sealing arterioles using the electroadhered cationic hydrogel.

13. The method of claim 2 further comprising measuring the gel-tissue adhesion strength between the anionic hydrogel and/or group of anionic cells and the cationic hydrogel by sticking samples onto glass slides using a cyanoacrylate glue.

14. A system for accomplishing sutureless tissue repair comprising:
a cationic hydrogel;
anionic cells; and
electrodes adapted to (1) receive power from a direct current (DC) power supply; and (2) contact said cationic hydrogel and said anionic cells.

15. The system of claim 14 wherein the anionic hydrogel is formed into a cylindrical tube.

16. The system of claim 14 wherein the cationic hydrogel is formed into a strip.

17. An electroadhered material comprising:
a covalently crosslinked gel electroadhered to a physically crosslinked gel.

18. The electroadhered material of claim 17 wherein the covalently crosslinked gel comprises alginate crosslinked by divalent Ca²⁺ cations.

19. The electroadhered material of claim 17 wherein the physically crosslinked gel comprises a mixture that includes a nonionic monomer, a cationic monomer, a nonionic crosslinker, and crystalline nanoparticles having a highly ionic surface area.

20. The electroadhered material of claim 18 wherein the nonionic monomer comprises acrylamide.

21. The electroadhered material of claim 18 wherein the cationic monomer comprises quaternized dimethyl aminoethyl methacrylate.

22. The electroadhered material of claim 18 wherein the nonionic crosslinker comprises bis(acrylamide).

23. The electroadhered material of claim 18 wherein the crystalline nanoparticles with the ionic surface area comprise laponite nanoparticles.
